SEQUENCE LISTING (6 sequences)

SEQ ID NO: 1 - DNA, 15 bp, Artificial Sequence
  Description of Sequence cDNA from RefSeq database
gtaaccactgccgtg

SEQ ID NO: 2 - DNA, 15 bp, Artificial Sequence
  Description of Sequence a part of Human Genome Sequence from RefSeq database
gtatccacttccgtc

SEQ ID NO: 3 - DNA, 18 bp, Artificial Sequence
  Description of Artificial Sequence cDNA from RefSeq database
gtaaccactgccgtgtcc

SEQ ID NO: 4 - DNA, 18 bp, Artificial Sequence
  Description of Artificial Sequence a part of Human Genome Sequence from RefSeq database
gtatccacttccgtctcc

SEQ ID NO: 5 - DNA, 10 bp, Artificial Sequence
  Description of Artificial Sequence a part of Human Genome Sequence from RefSeq database
agcgtaagtg

SEQ ID NO: 6 - DNA, 10 bp, Artificial Sequence
  Description of Artificial Sequence a part of Human Genome Sequence from RefSeq database
tctctagcaa

(19) United States
(12) Patent Application Publication
(10) Pub. No.: US 2005/0159898 A1
(43) Pub. Date: Jul. 21, 2005

(54) Method that aligns cDNA sequences to genome sequences

(75) Inventors: Tomohiro Yasuda, Tokyo (JP); Toru Hisamitsu, Oi (JP)

Correspondence Address: Stanley P. Fisher;Reed Smith LLP, 3110 Fairview Park Drive, Suite 1400, Falls Church, VA, 22042-4503, US

(73) Assignee: Hitachi, Ltd.

(21) Appl. No.: 11/011,954

(22) Filed: Dec. 15, 2004

(30) Foreign Application Priority Data
Dec. 19, 2003 (JP) 2003-423065

(51) Int. Cl. C12Q 1/68; G06F 19/00; G01N 33/48; G01N 33/50

(52) U.S. Cl. 702/20

(57) ABSTRACT

Method and apparatus for mapping cDNA sequences to genome sequences at high speed are disclosed. A genome sequence is divided into K-base-length partial sequences that do not overlap and are continuous (K-mers). Then, they are stored in a table with coordinates on the genome sequence where each of them appears. Using this table, correspondences of K-mers are created from perfectly matching pairs of K-mers on the cDNA and the K-mers on the genome sequence. Of all the correspondences of K-mers, those sets that represent correct mapping rather than accidental coincidence are identified at high speed by a method based on a publicly known method that extracts a longest increasing partial sequence from a numerical sequence. The resultant correspondences of K-mers are extended to the association between bases by sequence alignment, and then correction at splice sites is performed. In order to allow for an optimum selection of parameters, an interactive interface capable of real-time response is provided.

### CLAIM OF PRIORITY

The present application claims priority from Japanese application JP 2003-423065 filed on Dec. 19, 2003, the content of which is hereby incorporated by reference into this application.

### BACKGROUND OF THE INVENTION

1. Field of the Invention

The present invention relates to a method of mapping cDNA sequences onto genome sequences at high speed.

2. Description of Related Art

In June 2000, the International Human Genome Sequencing Consortium and Celera Genomics of the U.S. have announced that they have completed the determination of the draft sequence of the human genorne, and it is expected that a finished sequence will be produced no later than 2003 (International Human Genome Sequencing Consortium, Initial sequencing and analysis of the human genome, Nature, 409:860-921, 2001; Venter, J. C., et al., The sequence of the human genome, Science, 291:1304-1351, 2001). In order to obtain information that is unobtainable merely from the analysis of the genome sequence, the importance of cDNA sequence analysis is increasing, which is capable of directly analyzing the sequence of genes expressing in vivo. In Japan, a national project for acquiring the human cDNA sequence called Full-Length Human cDNA Sequencing Project (http://www.nedo.go.jp/bio-e/) was underway for three years until 2001, and similar projects are being undertaken in the U.S. and Germany (Strausberg, R. L., Feingold, E. A., Klausner, R. D., Collins, F. S., The Mammalian Gene Collection, Science, 286:455-457, 1999; Wiemann, S., et al., Toward a Catalog of Human Genes and Proteins: Sequencing and Analysis of 500 Novel Complete Protein Coding Human cDNAs, Genome Res., 11(3):422-435, 2001).

Identifying the location of a cDNA sequence on a genome sequence and clarifying the correspondence between the cDNA sequence and the genome sequence on a base-by-base basis, namely the mapping of the cDNA sequence onto the genome sequence, is important for the elucidation of biological phenomena for the following reasons. First, because a cDNA sequence is the very sequence of a gene that is being expressed, it helps identify the region on the genome sequence that corresponds to the gene, and it also helps learn the location of a specific gene of interest on the genome. By clarifying the location of a gene on the genome, it also becomes possible to analyze promoter sequences that control the expression of the gene. Furthermore, while it is difficult to identify the exon/intron structure of a gene by simply analyzing the genome sequence or the cDNA sequence individually, an accurate identification can be performed by mapping the cDNA sequence onto the genome sequence.

The volume of cDNA sequences that are stored in public databases and published is ever increasing. In the Full-Length Human cDNA Sequencing Project, 20,894 sequences with an average length of about 2.3 kbp were determined (as tallied by Helix Research Institute, Inc., and the Institute of Medical Science, the University of Tokyo). The volume of data on sequences called ESTs, which is a partial sequencing of cDNA sequences, in the dbEST database from NCBI of the U.S. is more than five million sequences for humans alone (Boguski, M. S., Lowe, T. M., Tolstoshev, C. M., dbEST—database for “expressed sequence tags”, Nat. Genet., 4(4):332-3,1993). Meanwhile, the genome sequence is a gigantic sequence composed of about three billion bases. In order to allow for the input of such huge sequence data and carry out mapping, a system capable of processing large-scale sequence data at high speed is needed.

Known examples of the tools useful for the mapping of cDNA sequences to genome sequences include BLAST (Altschul, S. F., Madden, T. L., Schaffer, A. A., Zhang, J., Zhang, Z., Miller, W., and Lipman, D. J., Gapped BLAST and PSI-BLAST: a new generation of protein database search programs, Nuc. Acid Res. 25:3389-3402, 1997), MegaBLAST (Zhang, Z., Schwartz, S., Wagner, L., and Miller, W., A Greedy Algorithm for Aligning DNA Sequences, J. Comput. Biol., 7:203-214, 2000), sim4 (Florea, L., Hartzell, G., Hang, Z., Rubin, G. M., and Miller, W., A Computer Program for Aligning a cDNA Sequence with a Genomic DNA Sequence, Genome Res., 8:967-974, 1998), BLAT (Kent, J. W., BLAT—The BLAST-like Alignment Tool, Genome Res., 12:656-664, 2002), and Squall (Ogasawara, J. and Morishita, S., Fast and Sensitive Algorithm for Aligning ESTs to Human Genome, Proceedings of the IEEE Computer Society Bioinformatics Conference, 2002).

BLAST and MegaBLAST are general software that search a database for sequences similar to a query sequence. Since these technologies were not developed for the purpose of mapping to genome sequences, they do not take into consideration the exon/intron structure of genes or the fact that intron sequences in many cases start with GT and end with AG. Therefore, they cannot be used for mapping as is, and it is indispensable to develop a post-processing system for performing processes necessary for mapping.

As a mapping tool that takes the exon/intron structure of genes, for example, into consideration, sim4 is often utilized. However, according to a study published in Ogasawara, J. and Morishita, S., Fast and Sensitive Algorithm for Aligning ESTs to Human Genome, Proceedings of the IEEE Computer Society Bioinformatics Conference, 2002, sim4 is seven times slower than BLAT and 400 times slower than Squall, both of which were developed more recently. Thus, it is difficult to use sim4 for the annotation of large-scale sequence information.

BLAT, which was developed at the University of California, Santa Cruz, is a tool reputed for its processing speed that is capable of operating even in a meager main-memory, inexpensive computing environment. It is, however, not so fast as Squall, which is described in the following.

The processing speed of Squall, which was developed at the University of Tokyo, far exceeds that of BLAT. But Squall requires a large-capacity main memory and is believed to be only operable on a large-scale computer if a large-scale genome, such as the human genome, has to be dealt with.

A patent application relating to the mapping of cDNA sequences to genome sequences filed by RIKEN is also pending (JP Patent Publication (Kokai) No. 2001-155009 A: “Apparatus for determining exon/intron junctions, apparatus for determining gene regions, and methods therefor,” Yoshihide Hayashizaki, inventor, of RIKEN). However, this technology depends on an external program such as BLAST for performing the processes for the search for similar regions between a cDNA sequence and the genome sequence. Namely, the technology covers only part of the entire mapping process.

### SUMMARY OF THE INVENTION

For the purpose of discussing the problems to be solved when mapping cDNA sequences to genome sequences, the correspondence between cDNA sequences and genome sequences will be described.

A gene on a genome is first transcribed to a pre-mRNA, as shown in FIG. 2, which is followed by a process called splicing in which only regions called exons are left, thereby producing a mRNA. The regions that are removed in this process are called introns. Because mRNAs are unstable substance that can be easily broken, they are in many cases converted into DNA in a process called reverse transcription when performing an analysis such as sequencing. The resultant DNA is cDNA (complementary DNA). Thus, a cDNA sequence can be thought of as a part of the genome sequence from which some parts have further been eliminated. However, because the cDNA sequence and the genome sequence are not determined for the same individual, there are variations due to differences between individuals. There could also be variations due to sequencing error.

Thus, in order to allow the cDNA sequence to be mapped to the genome sequence at high speed, it is necessary to identify the location where the exon portions of the cDNA sequence and the genome sequence are similar, to compare the cDNA sequence and the genome sequence and align them while permitting sequence variations to some extent, and to identify exon boundaries in the cDNA sequence by comparing it with the genome sequence.

In accordance with the invention, a cDNA sequence is mapped in the following steps:

(1) The genome sequence is disassembled into a partial string of K bases that do not overlap, i.e., non-overlapping K-mers. Then, the K-mers are registered in a table with the locations on the genome where the K-mers appear.

(2) When a Kmer at a location p on a cDNA sequence perfectly matches a K-mer taken from a location q on a genome sequence in the table, a pair of the values of p and q, (p, q), is created.

(3) A sequence S(p) is created by arranging a sequences of the entire pairs (p, q) regarding the K-mers at location p on the cDNA sequence in the decreasing order with respect to q. S(p) may be a sequence with zero elements.

(4) Each S(p) is connected in the increasing order of p to create a sequence S of pairs, namely S=S(0)S(1)S(2) . . . S(n-1), where n is the number of the overlapping K-mers on the cDNA sequence.

(5) A partial sequence S′ is extracted from S, where the value of q in S′ must be in the increasing order, and S′ must be the longest one of the partial sequences with the increasing order of q.

(6) The sequence S′ of pairs is read from the beginning, and a correspondence of a K-mer at location p on the cDNA sequence and a K-mer at location q on the genome sequence is selected upon appearance of a pair (p, q). Correspondences of K-mers that have not been selected at the end of reading of S′ are disclosed.

(7) The correspondences between K-mers obtained as a result of the above processes are extended to correspondences of bases on the sequences according to the character string comparison method described in Zhang, Z., Schwartz, S., Wagner, L., and Miller, W., A Greedy Algorithm for Aligning DNA Sequences, J. Comput. Biol., 7:203-214, 2000. After that, the alignment is corrected so that the intron sequence begins with GT and ends with AG.

In accordance with the invention, it becomes possible to map cDNA sequences to genome sequences at high speed on a small-scale computer system, such as a personal computer.

### BRIEF DESCRIPTION OF THE DRAWINGS

FIG. 1 illustrates the process of selecting from perfectly matching correspondences of K-mers on a cDNA sequence and K-mers on a genome sequence those correspondences consistent with mapping, in accordance with the method of the invention.

FIG. 2 illustrates the relationship between a genome and a cDNA, and the concept of mapping.

FIG. 3 schematically shows the method of the invention.

FIG. 4 illustrates a process of registering non-overlapping K-mers on a genome sequence in a table, where K=3.

FIG. 5 illustrates a process of registering overlapping K-mers on a genome sequence in a table, where K=3.

FIG. 6 illustrates perfectly matching correspondences of K-mers on a cDNA and K-mers on a genome sequence.

FIG. 7 illustrates a process of extending the correspondences between K-mers to the alignment of bases on the basis of sequence comparison.

FIG. 8 illustrates a possible event in which, when expanding the correspondences between K-mers into the alignment of bases based on sequence comparison, there is more than one association between K-mers, resulting in redundancy in the process of sequence comparison.

FIG. 9 illustrates that introns begins with GT and ends with AG in many cases.

FIG. 10 illustrates a process of correcting the association between bases at splice sites so that the intron starts with GT and ends with AG.

FIG. 11 shows an example of an interface that enables the parameters of the invention to be adjusted while users examine the result of mapping in real time.

FIG. 12 shows an example of an apparatus for implementing the interface of FIG. 11.

FIG. 13 shows an example of a dialogue for specifying sequences and parameters to be provided to the method of the invention.

FIG. 14 shows a flowchart of a process of associating the K-mers on the cDNA sequence with the K-mers on the genome sequence in accordance with the method of the invention.

FIG. 15 shows a flowchart illustrating the association between the processes performed by the hardware of the interface and software during an automatic, batch execution of the mapping process according to the method of the invention.

FIG. 16 shows a flowchart illustrating the association between the processes performed by the hardware of the interface and software when performing a mapping process through an interactive interface in accordance with the method of the invention.

### DETAILED DESCRIPTION OF THE PREFERRED EMBODIMENTS

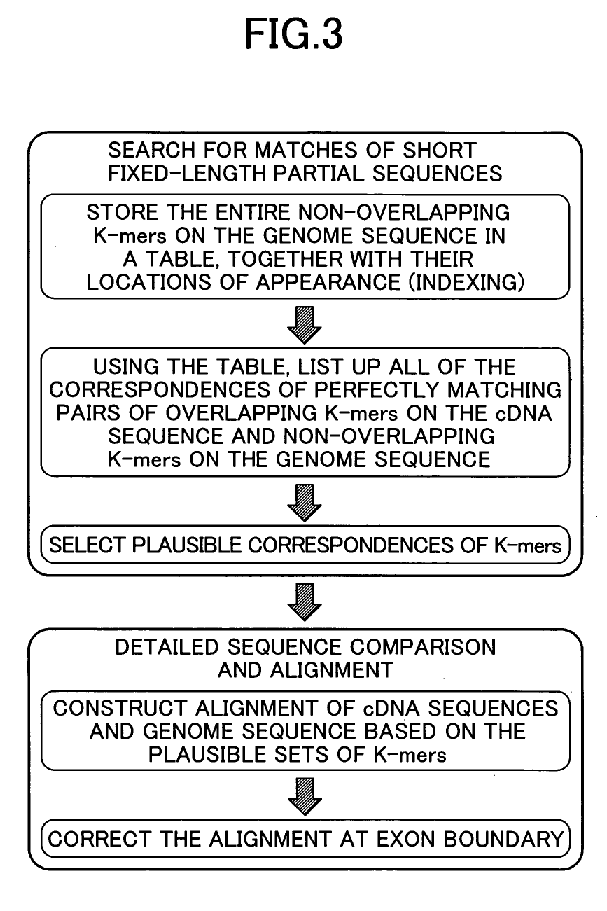

The outline of the method of the invention is shown in FIG. 3. The term “K-mer” herein refers to a short base sequence with a length of K bases, where K is a parameter specified by the user, which is recommended to be about 30 bases at most.

(Indexing of the Genome Sequence)

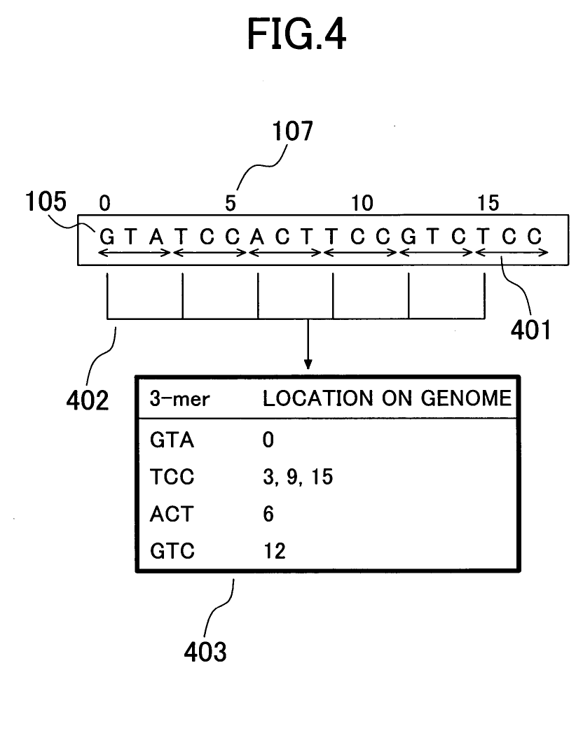

Initially, the location on the genome sequence where each K-mer appears is registered in a table. In accordance with the invention, not all K-mers on the genome sequence but a single K-mer for every K bases is recorded in the table so that adjacent K-mers do not overlap. FIG. 4 shows an example where K=3. K-mers at more locations in the genome sequence than a parameter given by the user are considered to be a part of a repeat sequence and disregarded in the subsequent processes. In the example of FIG. 4, if the user parameter concerning the number of times of appearance is two, “TCC,” which appears at three locations, is disregarded in the subsequent processes.

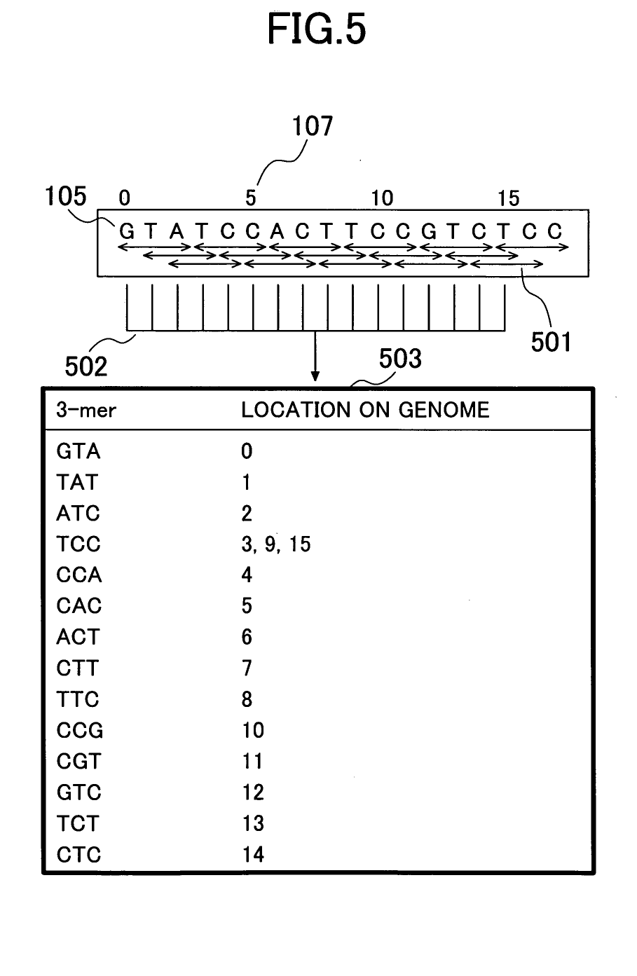

There are two methods for indexing K-mers on the genome sequence. One involves the extraction of K-mers for every K bases on the genome sequence, namely a method employing non-overlapping K-mers, as shown in FIG. 4. The other involves the registration of every K-mer on the genome sequence in a table, namely a method employing overlapping K-mers, as shown in FIG. 5. While the method using non-overlapping K-mers is disadvantageous in that there is a high possibility of overlooking the matching of K-mers in the event of a sequence error, it has the advantage that the amount of main memory required in the computer can be reduced to about 1/K as compared with the method using overlapping K-mers. For this reason, the indexing method based on non-overlapping K-mers has been adopted in the invention which requires less main memory.

(Listing of Correspondences of Perfectly Matching K-mers on the cDNA Sequence and the Genome Sequence)

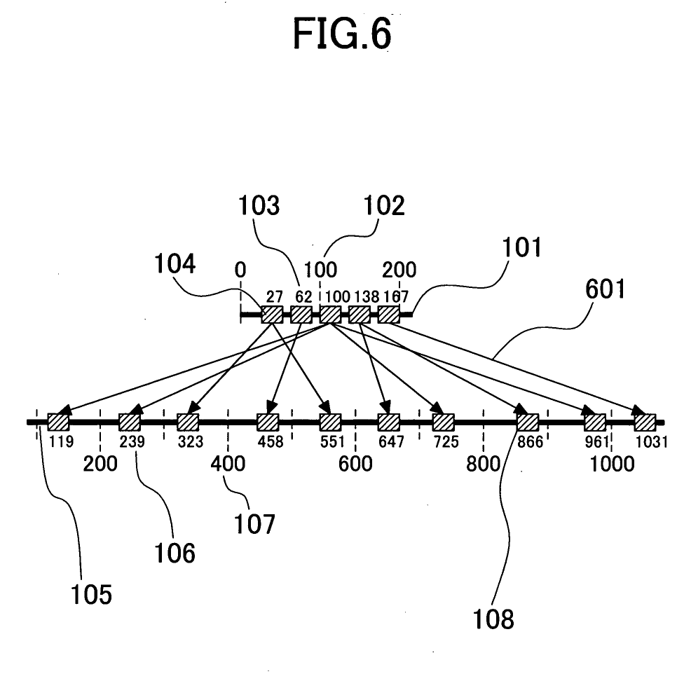

For each of the K-mers on the cDNA sequence, the aforementioned table is searched for those K-mers that perfectly match. Then, resultant correspondences of K-mers are listed with their locations. Then, K-mers on the cDNA sequence are presumed to be overlapping K-mers. Since the K-mers on the genome sequence registered in the table are non-overlapping K-mers, it can be expected that, except for the exon boundary, the K-mers on the cDNA sequence will perfectly match the K-mers on the genome sequence for every K bases. However, due to variations arising from SNPs or the like, there could be cases where the K-mers do not perfectly match between the cDNA sequence and the genome sequence at locations other than exon boundaries. On the other hand, there could also be cases where a perfect match of K-mers is observed that is irrelevant to the location of the gene on the genome sequence, as a result of an accidental matching of the sequences (FIG. 6).

(Selection of Plausible Correspondences of Perfectly Matching K-mers)

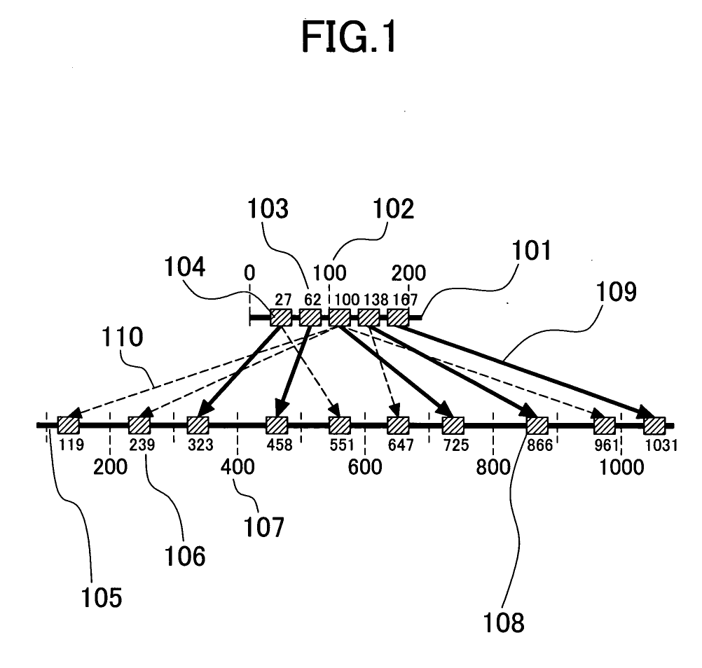

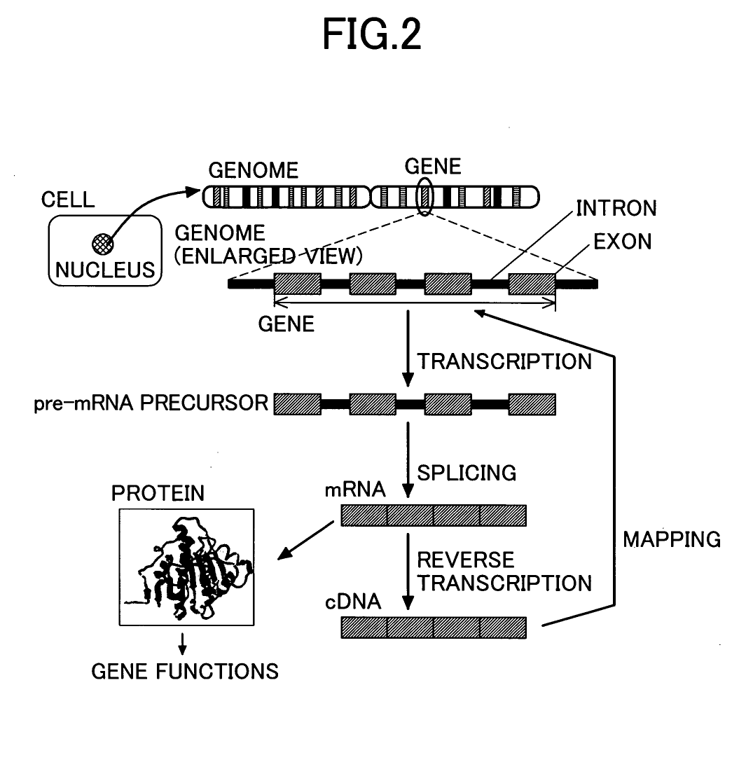

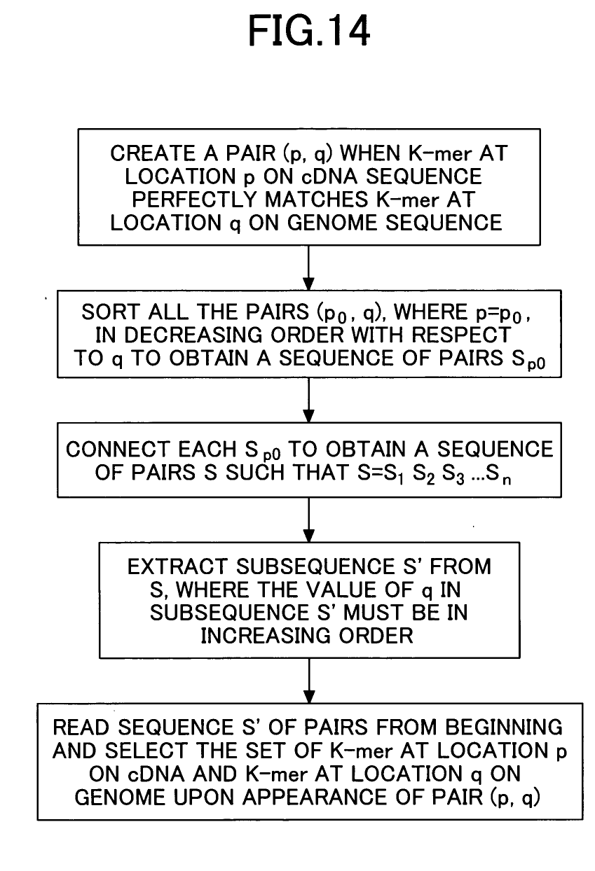

It is necessary to select only correspondences of K-mers that seem plausible, from all correspondences including an accidental matching, of the perfectly matching K-mers on the cDNA sequence and the genome sequence, as indicated by bold arrows 109 in FIG. 1. In accordance with the invention, attention was focused on the fact that, in the case of identical strands, the K-mer located upstream of a cDNA sequence is also located upstream on a genome sequence. The outline of the method of the invention for selecting the plausible correspondences of perfectly matching K-mers is shown in FIG. 14.

In accordance with the method of the invention, a method for extracting the longest monotonically increasing subsequence of a given numerical sequence is utilized for the selection of K-mers. The problem of extracting the longest monotonically increasing subsequence of a given numerical sequence is called the “longest increasing subsequence problem”. The longest monotonously increasing sequence will be hereafter referred to as the longest increasing sequence (LIS). For example, <323, 458, 725, 866, 1031>is a LIS of a numerical sequence <551, 323, 458, 961, 725, 239, 119, 866, 647, 1031>. It is known that a LIS can be found within O(n log n)-time by a method described in Gusfield, D., Algorithms on strings, trees, and sequences. Computer Science and Computational Biology, Cambridge University Press, New York. Here, n is the length of the given numerical sequence. In the following, the method of the invention for selecting K-mers by applying the LIS-finding method will be described.

For any K-mer on location p on the cDNA sequence and any K-mer on location q on the genome sequence that perfectly match with each other, a pair (p, q) of the values of p and q is created. Then, a sequence composed of all the pairs (p, q) for a particular value of p is created and sorted in the decreasing order with respect to q, thereby obtaining a sequence S(p), which may be a sequence of zero elements. Each S(p) is connected in the increasing order of p to create a sequence S of pairs. Namely, S=S(0)S(1)S(2) . . . S(n-1), where n is the number of overlapping K-mers on the cDNA sequence.

From the thus created sequence S, a subsequence S′ is extracted. S′ must be the longest subsequence such that the value of the second element q monotonously increases. After extraction, such sequence S′ of pairs is read from the beginning, and, when a pair (p, q) appears, the correspondence of K-mers at location p on the cDNA sequence and at location q on the genome sequence is selected. The correspondences of K-mers that have not been selected after the entire S′ is read are discarded.

An example of the above-described method of selecting K-mers is described. A K-mer at location p=27 on the cDNA sequence corresponds to K-mers at locations q=323 and 551 on the genome. In the following, the circumstances in which a K-mer at p=62 perfectly matches a K-mer at q=458, a K-mer at p=100 perfectly matches K-mers at q=119, 239, 725, and 961, a K-mer at p=138 perfectly matches K-mers at q=647 and 866, and a K-mer at p=167 perfectly matches a K-mer at q=1031 will be discussed.

Initially, for each K-mer on the cDNA sequence, a list of pairs (p, q) is prepared and is sorted in the decreasing order of q. The sequences S(p) with the number of elements other than zero are the following five:
S (27)=<(27, 551), (27, 323)>
S (62)=<(62, 458)>
S (100)=<(100, 961), (100, 725), (100, 239), (100, 119)>
S (138)=<(138, 866), (138, 647)>
S (167)=<(167, 1031)>.

These sequences are then connected to produce a list S=S(0)S(1)S(2) . . . S(n-1). In the example of FIG. 6, S is as follows:
S=<(27, 551), (27, 323), (62, 458), (100, 961), (100, 725), (100, 239), (100, 119), (138, 866), (138, 647), (167, 1031)>.

Of all subsequences of S, the longest subsequence with monotonously increasing q is identified using the method that finds a LIS. In the example under analysis, the subsequence consisting of the elements enclosed by the brackets [] in the following expression is the longest subsequence of S with monotonously increasing q.

S=<(27, 551), [(27, 323), (62, 458)], (100, 961), [(100, 725)], (100, 239), (100, 119), [(138, 866)], (138, 647), [(167, 1031)]>

The aforementioned subsequence, S′, is thus:
S′=<(27, 323), (62, 458), (100, 725), (138, 866), (167, 1031)>.

Then, S′ is read from the beginning and, for each pair, the correspondence of perfectly matching K-mers on the cDNA sequence and the genome sequence is selected one by one. The K-mer at location p=27 on the cDNA sequence is made to correspond to the K-mer at location q=323 on the genome, and K-mers at p=62, 100, 138, and 167 on the cDNA sequence are made to correspond with K-mers at q=458, 725, 866 and 1031 on the genome, respectively. In this way, the plausible correspondences of perfectly matching K-mers are selected, as shown in FIG. 1.

Plausible correspondences of K-mers can be selected with this method for the following reasons. Since each of S(p) for any p is sorted in the decreasing order of q in step 2, the sequence of pairs corresponding to the same p in S is arranged in the decreasing order of the value of q. Thus, it is ensured that there is at most one pair corresponding to the same p at most in S′. Namely, an arbitrary K-mer on the cDNA sequence is mapped to at most one location on the genome with this method. Furthermore, since S′ is arranged in the increasing order of q in step 4, the location of K-mers with identical order on the cDNA sequence and the genome sequence is extracted. Of the sequences of K-mers with the increasing order of q, the longest is thought to be the most plausible.

Let n be the length of the sequence of K-mers obtained in this procedure, Q be the length of the cDNA sequence, and T be a parameter given by the user. Then, if nK/Q≧T is satisfied, it is thought that a sufficient number of K-mers on the cDNA sequence have been aligned to the K-mers on the genome sequence, namely, the cDNA sequence under investigation has been successfully mapped to the genome sequence.

In order to reduce the possibility that an accidentally produced correspondences of K-mers that satisfies nK/Q≧T with regard to the cDNA sequence that should not be mapped to the genome sequence, a window with a width of W bases is provided on the genome sequence in accordance with the invention, so that only those K-mers that are within the range of the window are processed. Adjacent windows are located to have an overlap of W/2 bases in order to avoid dividing any regions where the cDNA sequence is mapped at the window boundaries. If the number of K-mers within the window that perfectly match K-mers on the cDNA sequence is small and there is no prospect of nK/Q≧T being satisfied, that cDNA sequence is determined to be incapable of being mapped, and the K-mer selecting process is omitted. In this way, the process for calculating the LIS can be eliminated unless necessary, so that the overall processing time can be reduced.

(Alignment of the cDNA Sequence and the Genome Sequence)

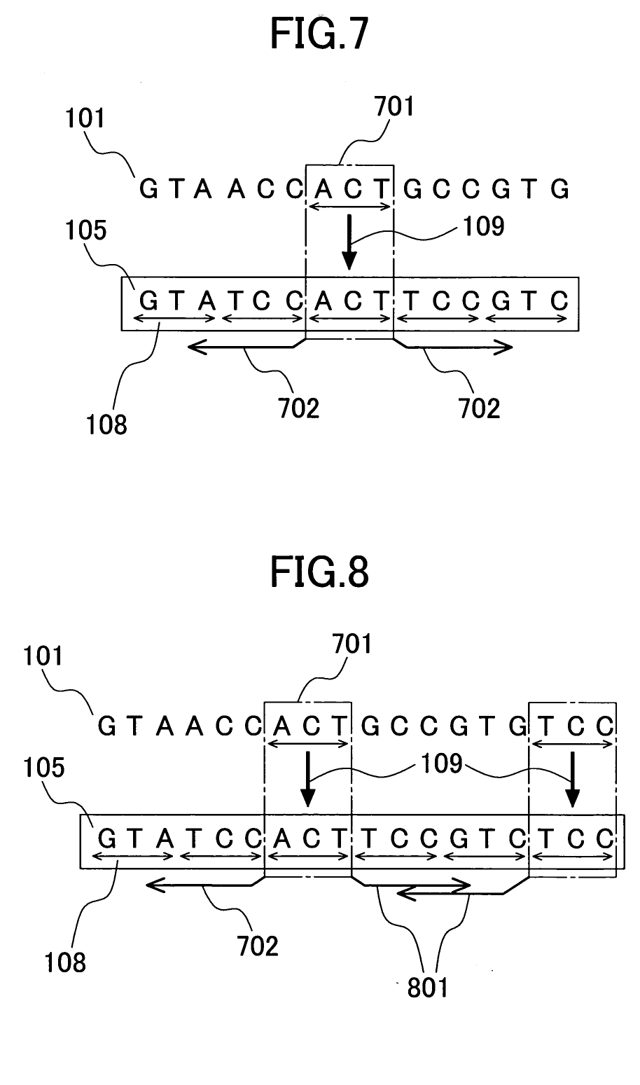

After those of the correspondences of perfectly matching K-mers on the cDNA sequence and the genome sequence that are plausible have been selected by the above-described procedure, the cDNA sequence and the genome sequence are compared to construct an alignment of base sequences (FIG. 7). The cDNA sequence and the genome sequence might not perfectly match even in exon regions, since some variations such as SNPs could be included. Therefore, a fast algorithm that permits variations in the sequences to some extent is required for the sequence comparison. One example of such an algorithm is described in Zhang, Z., Schwartz, S., Wagner, L., and Miller, W., A Greedy Algorithm for Aligning DNA Sequences, J. Comput. Biol., 7:203-214, 2000. During sequence comparison, if there are several correspondences of perfectly matching K-mers nearby, it is necessary to avoid carrying out the alignment process twice or more in the same region (FIG. 8). For that, the interval in which sequence comparison is carried out can be limited up to K-mers next to the current one and to the region that has already been aligned. If, as a result of sequence comparison, an aligned region abuts a region centered around an adjacent K-mer, they are regarded as a single exon and are integrated.

(Correction of Alignment At Splice Sites)

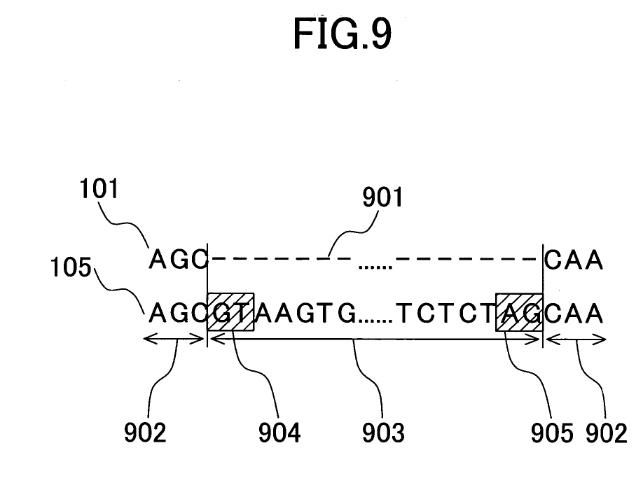

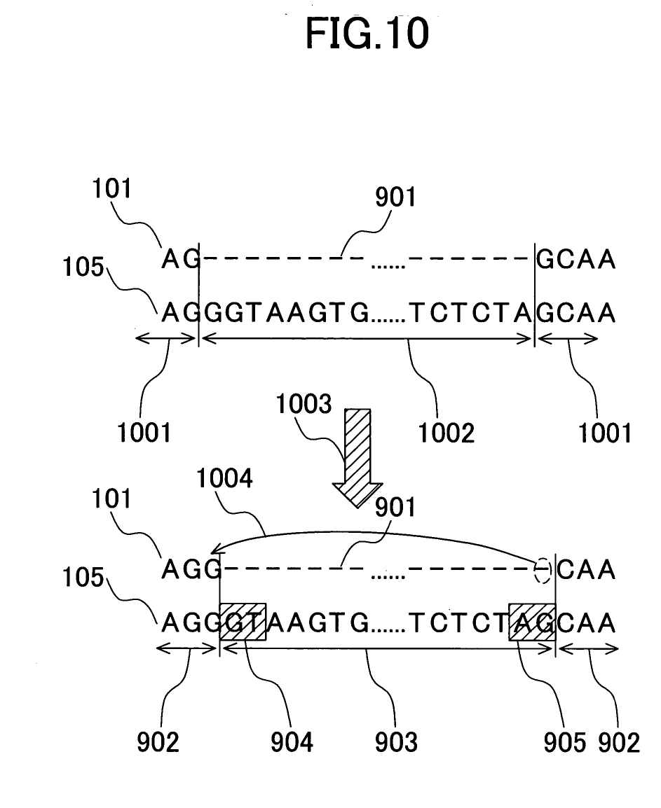

As shown in FIG. 9, an intron region on a genome in most cases starts with GT and ends with AG. According to a study by Burset et al., 98.71% follows this rule (Burset, M., Seledtsov, I. A., and Solovyev, V. V., SpliceDB: database of canonical and non-canonincal mammalian splice sites, Nuc. Acid. Res., 29:255-259, 2001). If there is ambiguity in alignment between a cDNA sequence and a genome sequence, as shown in FIG. 10, the location of the exon boundary is moved in order to configure the alignment so that the intron starts with GT and ends with AG, while avoiding introduction of mismatches, insertions or deletions. The bases at the start and end locations of an intron might in a small number of cases be GC-AG, instead of GT-AG. For this reason, if the GT-AG combination cannot be achieved by correction, attempts should preferably be made to configure the alignment that starts with GC and ends with AG by the same process.

(Analysis of the Statistical Significance of the Method of the Invention)

First, it will be shown that a cDNA sequence to be mapped to a genome sequence can be mapped by the method of the invention with high probability. Let M be the probability that a base of the cDNA sequence matches that of the genome sequence in their homogeneous areas, n be the number of K-mers that are mapped, N be the maximum value that n can take, and Q be the length of the cDNA sequence. When the probability of a cDNA sequence that can be mapped being judged to be capable of being mapped by the method of the invention is denoted by P(n≧QT/K), it satisfies the following equation:

$$P ( n \geq \frac{Q}{K} T ) = {\sum }_{\frac{Q}{k} T \leq i \leq N} ( \begin{matrix}N \\ i\end{matrix} ) {{p}^{i} ( 1 - p )}^{N - i} \qquad ( 1 )$$

where p=M^{K}.

Table 1 shows the results of calculation of P(n≧QT/K) in the case where Q=2000 in view of the fact that the length of full-length cDNA sequences is in many cases about 2000 bases, and where T=0.5.

TABLE 1

K

| | 8 | 9 | 10 | 11 | 12 | 13 | 14 |
|---|---|---|---|---|---|---|---|
| M = 0.91 | 0.1896 | 0.01812 | 0.000967 | 5.63E−05 | 1.60E−06 | 1.02E−07 | 2.94E−09 |
| M=0.92 | 0.685 | 0.2224 | 0.0363 | 0.004898 | 0.000338 | 3.81E−05 | 2.14E−06 |
| M=0.93 | 0.9748 | 0.7506 | 0.3508 | 0.1153 | 0.02082 | 0.004456 | 0.000529 |
| M=0.94 | 0.9998 | 0.988 | 0.8782 | 0.6249 | 0.2932 | 0.1259 | 0.03396 |
| M=0.95 | 1 | 1 | 0.9981 | 0.9779 | 0.8688 | 0.6887 | 0.4167 |
| M=0.96 | 1 | 1 | 1 | 1 | 0.9988 | 0.991 | 0.9489 |
| M=0.97 | 1 | 1 | 1 | 1 | 1 | 1 | 0.9999 |
| M=0.98 | 1 | 1 | 1 | 1 | 1 | 1 | 1 |
| M=0.99 | 1 | 1 | 1 | 1 | 1 | 1 | 1 |

If there are n correspondences of K-mers that are derived from correct mapping, at least n correspondences survive the process of selecting correct K-mers according to the invention. Therefore, if n≧QT/K is satisfied, it is judged that the cDNA sequence can be mapped according to the method of the invention. If M is 96% or more and K≦13, the cDNA sequence can be mapped with the probability of 99% or more. In the calculation of Table 1, N was approximated by the largest integer not exceeding Q/K. The actual value of N depends on the number and locations of exon boundaries in the cDNA sequence and is somewhat smaller than Q/K in general. The size W of the window on the genome was assumed to be sufficient. The result of analysis with the technology described in Kent, J. W., BLAT—The BLAST-like Alignment Tool, Genome Res., 12:656-664, 2002, shows that, when the sequences in the RefSeq database (Pruitt, K. D. and Maglott, D. R., RefSeq and LocusLink: NCBI gene-centered resources, Nuc. Acid. Res., 29:137-140, 2001) are mapped to genome sequences, the width of the mapped region on the genome sequences is about 2.3 million bases at maximum, indicating that the size W of the window is enough if it is several million base large.

Hereafter it is shown that the probability of nK/Q≧T being satisfied due to accidental matches is small. The expression (2) shows the expected number of times that accidental matches of K-mers occur within a window of a width W on a genome sequence with respect to a cDNA sequence of a length of Q bases, as discussed in Kent, J. W., BLAT—The BLAST-like Alignment Tool, Genome Res., 12:656-664, 2002.:

$$( Q - K + 1 ) \cdot \frac{W}{K} \cdot {( \frac{1}{A} )}^{K} \qquad ( 2 )$$

Table 2 shows the actually calculated values for a plurality of W and K.

TABLE 2

| | K: | | | | | | |
|---|---|---|---|---|---|---|---|
| | 8 | 9 | 10 | 11 | 12 | 13 | 14 |
| | Q/K: | | | | | | |
| | 250 | 222 | 200 | 181 | 166 | 153 | 142 |
| | (Q/K) * T | | | | | | |
| | 125 | 111 | 100 | 90 | 83 | 76 | 71 |
| W = | | | | | | | |
| 1.00E+06 | 3801.35 | 844.32 | 189.877 | 43.1321 | 9.87947 | 2.27873 | 0.528725 |
| 2.00E+06 | 7602.69 | 1688.64 | 379.753 | 86.2642 | 19.7589 | 4.55746 | 1.05745 |
| 3.00E+06 | 11404 | 2532.96 | 569.63 | 129.396 | 29.6384 | 6.83619 | 1.58618 |
| 4.00E+06 | 15205.4 | 3377.28 | 759.506 | 172.528 | 39.5179 | 9.11493 | 2.1149 |
| 5.00E+06 | 19006.7 | 4221.6 | 949.383 | 215.66 | 49.3973 | 11.3937 | 2.64363 |
| 6.00E+06 | 22808.1 | 5065.92 | 1139.26 | 258.793 | 59.2768 | 13.6724 | 3.17235 |
| 7.00E+06 | 26609.4 | 5910.24 | 1329.14 | 301.925 | 69.1563 | 15.9511 | 3.70108 |
| 8.00E+06 | 30410.8 | 6754.56 | 1519.01 | 345.057 | 79.0358 | 18.2299 | 4.2298 |
| 9.00E+06 | 34212.1 | 7598.88 | 1708.89 | 388.189 | 88.9152 | 20.5086 | 4.75853 |
| 1.00E+07 | 38013.5 | 8443.2 | 1898.77 | 431.321 | 98.7947 | 22.7873 | 5.28725 |

The values of expected numbers of accidental matches are shown in Table 2. Since they are average values, a greater number of accidental matches could occur. It will be shown hereafter that the possibility of nK/Q≧T being satisfied in such a case is almost nil if parameters are appropriately selected. In general, it is known (Rains, E. M., Increasing subsequences and the classical groups, Electr. J. Com. 5(1), 1998) that the length Ln of a longest increasing subsequence that exists in a random permutation with a length n follows a probability distribution indicated by the following equation:

$$P ( {L}_{n} \leq k ) = \frac{{2}^{2 n} n !}{{( 2 π )}^{k} k ! ( 2 n ) !} {\int }_{- π}^{π} … {\int }_{- π}^{π} {( {\sum }_{1 \leq j \leq k} cos ( {θ}_{j} ) )}^{2 n} {\prod }_{1 \leq j \neq l \leq k} \vert {ⅇ}^{i {θ}_{j}} - {ⅇ}^{i {θ}_{l}} \vert ⅆ {θ}_{1} … ⅆ {θ}_{k} \qquad ( 3 )$$

However, since it is difficult to calculate the above equation directly, the probability of Ln equaling or exceeding length k is evaluated herein in accordance with the following inequality, which provide the upper limit of the probability.

$$P ( {L}_{n} \geq k ) \leq \frac{1}{k !} \cdot \frac{n !}{k ! ( n - k ) !} = \frac{1}{k !} ( \begin{matrix}n \\ k\end{matrix} ) \qquad ( 4 )$$

The grounds on which equation (4) is based are that, when Ln≧k, at least one increasing subsequence with length k or longer exists, that the number of subsequences with length k is n!/(k! (N-k)!), and that the probability of each of the subsequences being an increasing sequence is 1/k! each.

Table 3 shows the upper limit of the probability that nK/Q≧T holds (the value of the right-hand side of inequality (4)) in the case where there are three times more instances of perfectly matching K-mers than average. When the magnitude of dispersion is taken into consideration, it can be assumed that there is hardly any case in practice where the number of perfectly matching K-mers is three times more than average.

TABLE 3

| | K: | | | | | | |
|---|---|---|---|---|---|---|---|
| | 8 | 9 | 10 | 11 | 12 | 13 | 14 |
| | Q/K: | | | | | | |
| | 250 | 222 | 200 | 181 | 166 | 153 | 142 |
| | (Q/K) * T | | | | | | |
| | 125 | 111 | 100 | 90 | 83 | 76 | 71 |
| W = | | | | | | | |
| 1.00E+06 | 1.10E+28 | 1.10E−39 | 3.54E−103 | 0 | 0 | 0 | 0 |
| 2.00E+06 | 1.32E+66 | 1.38E−04 | 4.76E−65 | 0 | 0 | 0 | 0 |
| 3.00E+06 | 1.93E+88 | 1.70E+16 | 3.56E−45 | 1.10E−105 | 0 | 0 | 0 |
| 4.00E+06 | 9.48E+103 | 2.40E+30 | 1.32E−31 | 2.03E−88 | 0 | 0 | 0 |
| 5.00E+06 | 1.36E+116 | 1.97E+41 | 2.74E−21 | 1.12E−76 | 0 | 0 | 0 |
| 6.00E+06 | 1.16E+126 | 1.54E+50 | 5.85E−13 | 1.02E−67 | 0 | 0 | 0 |
| 7.00E+06 | 2.83E+134 | 5.04E+57 | 5.66E−06 | 1.87E−60 | 0 | 0 | 0 |
| 8.00E+06 | 5.21E+141 | 1.56E+64 | 5.87E+00 | 3.27E−54 | 0 | 0 | 0 |
| 9.00E+06 | 1.33E+148 | 8.21E+69 | 1.06E+06 | 5.99E−49 | 9.93E−118 | 0 | 0 |
| 1.00E+07 | 7.13E+153 | 1.08E+75 | 5.45E+10 | 2.58E−44 | 4.63E−108 | 0 | 0 |

From Table 3, it can be seen that, with regard to the aforementioned parameters, there is hardly any case where nK/Q≧T is satisfied accidentally when K=10 and W≦7.00E+06, or K≧11. Since P(Ln≧k)≦P(Ln′≧k) (n′≧n), even when the case of a much smaller number of perfectly matching correspondences is taken into consideration, the probability that those correspondences accidentally satisfy the conditions for mapping is very small.

Embodiment 1

A prototype system was constructed in which the method of the invention was implemented, and it was examined whether human cDNA sequences in the RefSeq database (Pruitt, K. D. and Maglott, D. R., RefSeq and LocusLink: NCBI gene-centered resources, Nuc. Acid. Res., 29:137-140, 2001) can be correctly judged whether each of them is derived from genes on chromosome 22 by mapping them to the genome sequence of the chromosome 22. For the RefSeq sequences, those uploaded on Jan. 26, 2003 were used. The chromosome from which each of human cDNA sequences in the RefSeq database is derived from is known. There were 453 sequences derived from chromosome 22.

First, it was evaluated whether human cDNA sequences in the RefSeq database derived from chromosome 22 can be correctly mapped to the genome sequence of the chromosome 22. As a result, only seven could not be mapped out of the 453 sequences. Therefore, (453-7)/453=98.5% of the cDNA sequences was successfully mapped.

Meanwhile, attempts were made to map the entire human cDNA sequences in the RefSeq database to the genome sequence of the chromosome 22, and it was evaluated if there were any sequences that were erroneously mapped. As a result, of the entire 18,255 sequences, 504 sequences were mapped to chromosome 22. Namely, (453-7)/504=88.5%, or nearly 90% of the mapped sequences was the cDNA sequences of chromosome 22.

Based on these results, it was confirmed that there was no serious problem in the mapping of the cDNA sequences to the genome sequence. Note that a sequence not derived from the chromosome 22 might be mapped to the chromosome 22 in a case where it has high homology with a family gene, a paralog, or a pseudo-gene on the chromosome 22. Thus, the above 88.5% should be regarded as a lower, rather than the rate of accuracy per se.

In this experiment, the values of the parameters were K=12, T=0.40, and W=2×10⁶. As the base-sequence alignment algorithm, one disclosed in Zhang, Z., Schwartz, S., Wagner, L., and Miller, W., A Greedy Algorithm for Aligning DNA Sequences, J. Comput. Biol., 7:203-214, 2000 was employed. The correction of alignment at splice sites was only carried out with respect to GT-AG.

Embodiment 2

Kent, J. W., BLAT—The BLAST-like Alignment Tool, Genome Res., 12:656-664, 2002, and Ogasawara, J. and Morishita, S., Fast and Sensitive Algorithm for Aligning ESTs to Human Genome, Proceedings of the IEEE Computer Society Bioinformatics Conference, 2002 disclose techniques for mapping cDNA sequences to genome sequences at high speed, as the method of the invention. BLAT and Squall, which are systems implementing each of these techniques, are also widely known. The processing time required for the mapping of the entire RefSeq human cDNA sequences to the sequence of the chromosome 22 of the human genome was compared between these known systems and the aforementioned prototype system implementing the method of the invention. For the processing time in Squall and BLAT, the values disclosed in Ogasawara, J. and Morishita, S., Fast and Sensitive Algorithm for Aligning ESTs to Human Genome, Proceedings of the IEEE Computer Society Bioinformatics Conference, 2002, were cited. The prototype system of the invention is still in a stage where improvements must be made to enhance accuracy. However, the processes that consume the most of time in a mapping process, namely the identification of rough locations on the genome sequence that correspond to the cDNA sequences, and the process of sequence alignment, have already been incorporated in the system. Therefore, it is expected that there would be no significant drop in speed during the future improvements.

Table 4 shows the result of comparison of the processing time. Although there are differences in operating environment and the version of the RefSeq sequences used, it is still fair to say that the processing speed of the inventive prototype system far exceeds that of BLAT and is as fast as Squall, on the assumption that the processing speed is proportional to the clock frequency of CPU and that the calculation time per sequence does not depend on the version of the sequence. It should be noted that while Squall is comparable in processing speed to the system of the invention, it is thought that Squall consumes vast amounts of main memory of the computer because it has adopted overlapping K-mers for indexing. In contrast, the prototype system of the invention could successfully be operated on a personal computer with a main memory of mere 1 GB.

TABLE 4

| | Invention | Squall | BLAT | sim4 |
|---|---|---|---|---|
| Speed | 0.014 | 0.03 | 1.69 | 12 |
| (sec/sequence) | | | | |
| Computer | PC, | PrimePower | Same as | Same as on |
| system used | Pentium IV | 1000, | on the left | the left |
| | 1.7 GHz, | SPARC64, | | |
| | 1 GB RAM, | 675 MHz, | | |
| | Linux | 64 GB RAM, | | |
| | | Solaris 8 | | |
| Number of | 18255 | 14784 | 14878 | 14784 |
| sequences | | | | |
| processed | | | | |

Embodiment 3

While the optimum values of parameters can be estimated to some extent based on statistical evaluation, it is difficult to determine their optimum values in advance because they are also dependent on the input, namely the genome sequence and the cDNA sequence. In order to perform an accurate mapping, it is desirable to use an interactive interface so that the user can adjust parameter values by monitoring the ongoing state of mapping. An example of such an interface is described below.

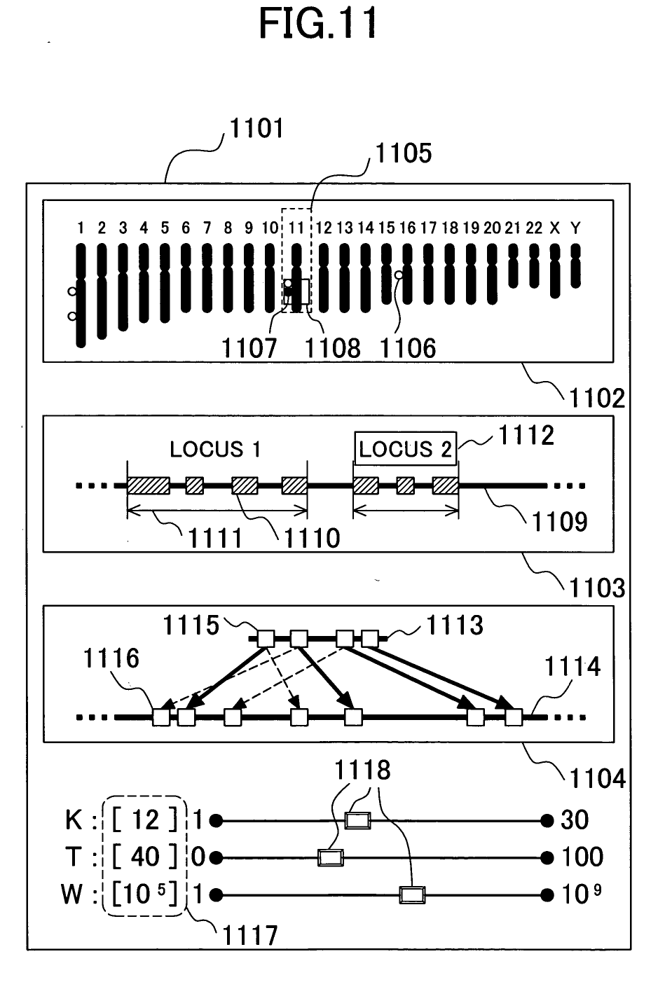

FIG. 11 shows an interface 1101 according to the invention. The interface 1101 is composed of an entire genome display area 1102, an enlarged genome display area 1103, a mapping state display area 1104, an input box 1117 for the display and entry of parameter values, and a slider 1118.

The entire genome display area 1102 provides a graphical display that symbolically shows entire genome sequences that are entered. In the example of FIG. 11, the entire autosomal chromosomes and sex chromosomes of the human genome are shown. In this entire genome display area 1102, there are displayed a graphical display 1105 that highlights a particular chromosome that is the subject of the enlarged genome display area 1103 and mapping state display area 1104, a mark 1106 indicating the area where the cDNA sequence is mapped, a mark 1107 indicating the location that is being displayed in the mapping state display area 1104, and a rectangle 1108 indicating the location corresponding to the area that is being displayed in the enlarged genome display area 1103.

When one of the graphical displays indicating chromosomes in the entire genome display area 1102 is clicked, the graphical display 1105 for highlighting the chromosome is moved to that chromosome. The thus highlighted chromosome becomes the subject of display in the enlarged genome display area 1103 and the mapping state display area 1104, and it also becomes the subject for which a mapping process is performed again when parameters are changed via 1117 or 1118. When the mark 1106 indicating the location where a cDNA sequence has been mapped is designated by a pointing device, the genome area displayed in the mapping state display area 1104 changes to any location of the mark 1106. Further, by changing the position of the rectangle 1108, the genome area that is displayed in the enlarged genome display area 1103 can be changed.

In the enlarged genome display area 1103, there is displayed part of a particular chromosome in an enlarged manner, thereby allowing the user to view an exon/intron structure obtained as a result of mapping. In the case where a part of the area being displayed in the enlarged genome display area 1103 is also displayed in the mapping state display area 1104, that part is highlighted by the rectangle 1112, for example, for ease of recognition.

In the mapping state display area 1104, there are displayed a graphical representation 1113 symbolically representing the cDNA sequence, a graphical representation 1114 symbolically representing the genome sequence, a graphical representation 1115 symbolically representing the K-mers on the cDNA sequence, and a graphical representation 1116 symbolically representing the K-mers on the genome sequence.

The values of parameters K, T, and W can be changed by value input boxes 1117 or by sliders 1118. When adjusting the value of K, however, indexing of the genome sequence by the non-overlapping K-mers should have been completed in advance for all of the values that K can have. In a case where it is difficult to maintain a table of the result of indexing by non-overlapping K-mers for a plurality of Ks, due to such factors as a limited main memory volume, the value input box 1117 and the slider 1118 are fixed to a single value for K so that no change of K can be accommodated.

While the sensitivity of mapping can be improved by decreasing the value of the parameter T, the chances of correspondences of K-mers accidentally satisfying nK/Q≧T increases, resulting in greater noise. Similarly, the accuracy of mapping of a gene with a long locus can be improved by increasing W, this might lead to an increase in noise. Further, while an improvement in sensitivity can be expected by decreasing the value of K, which would make perfectly matching K-mers less vulnerable to the influence of SNPs, for example, this might result in an increase in noise, as in the case of decreasing T with increasing W. The user can select optimal parameter values by changing the values of K, T, and W using the interface 1101 while monitoring the display areas 1102, 1103, and 1104.

In order to render the above-described interactive interface, the recalculation of mapping and the updating of screen must be carried out in real time. In accordance with the method of the invention, mapping can be performed in 0.014 seconds per sequence in the case of chromosome 22, as shown in Table 4, which enables the interface to respond in real-time.

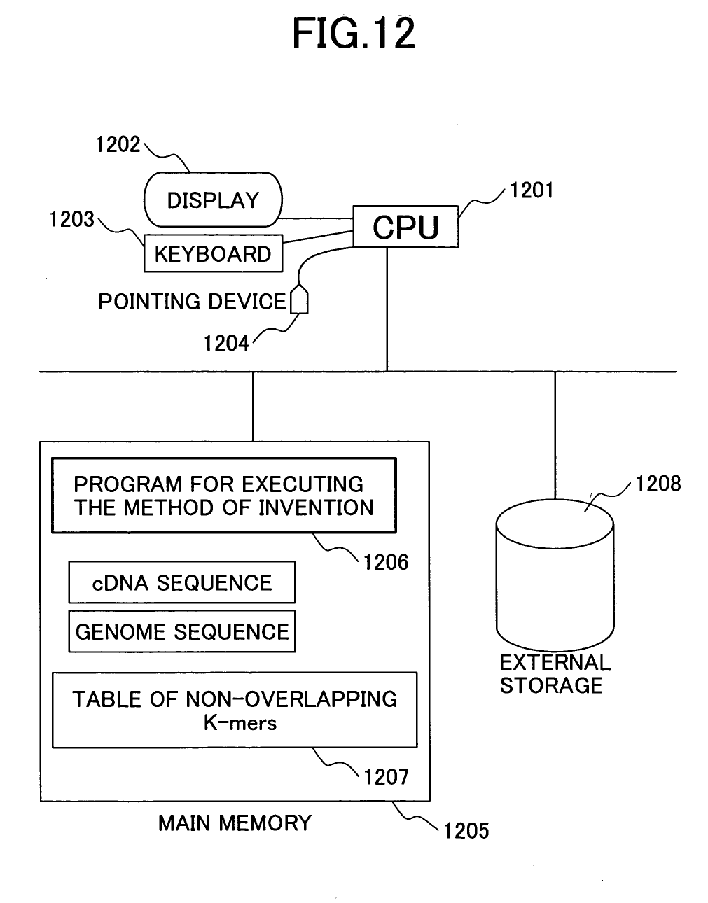

FIG. 12 shows an example of the configuration of the apparatus for implementing the above-described interface. The apparatus comprises a main memory 1205 which holds a program 1206 for carrying out the method of the invention, the cDNA sequence, and the genome sequence. The program 1206 is executed by a central processing unit 1201. The result of calculation in the interface 1101 shown in FIG. 11 is displayed on a display 1202. User inputs are made with a keyboard 1203 and a pointing device 1204.

(Execution of the cDNA Genome Mapping System)

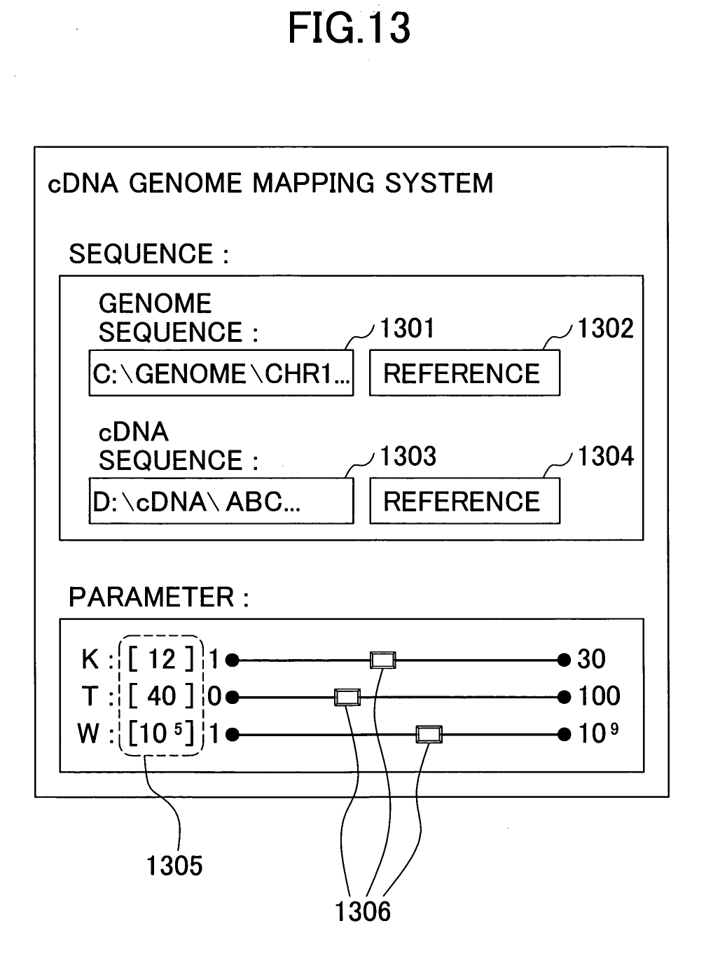

FIG. 13 shows an example of an initial screen for executing the cDNA genome mapping system of the invention on a terminal. It is a GUI interface for the designation of cDNA sequences, genome sequences, and parameters K, T, and W. In this exemplary interface, it is assumed that cDNA sequences and genome sequences are stored in files in external storages and that sequence data is obtained via a file name.

The file name of the file in which genome sequences are stored is either entered in an entry column 1301 via keyboard or the like, or designated using a file selector that can be caused to appear by pressing a button 1302. Similarly, the file name of the file in which cDNA sequences are stored is either entered in an entry column 1303 using keyboard or the like, or designated using a file selector that can be displayed by pressing a button 1304. The values of parameters K, T, and W are either entered directly in a corresponding portion of a value input box 1305 using keyboard or the like, or they can be changed using a slider 1306.

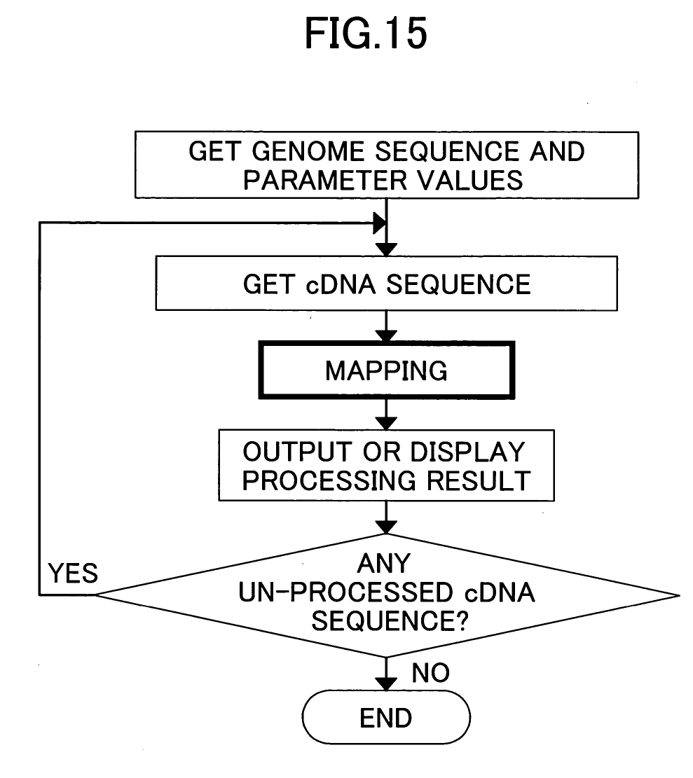

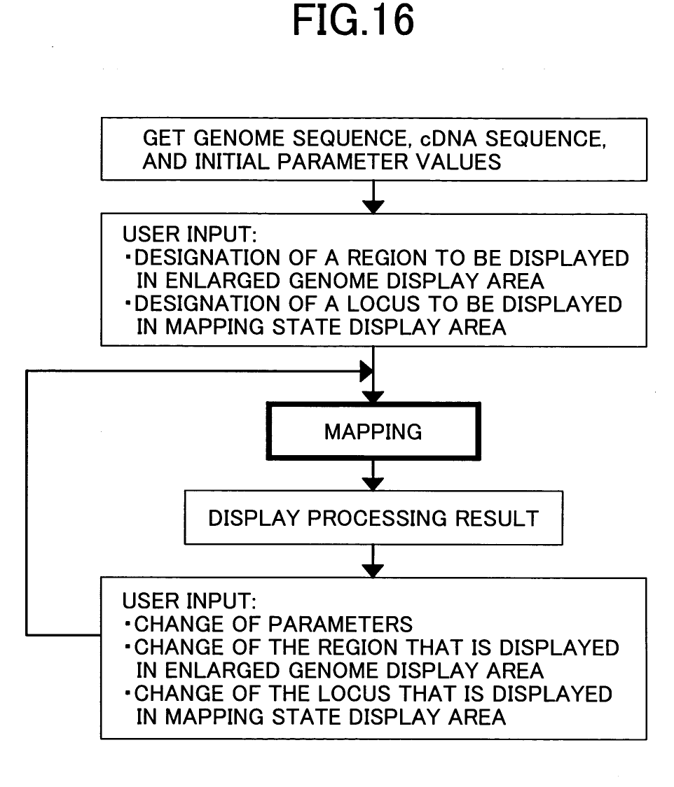

FIG. 15 shows a flowchart illustrating the association between the processes performed by the hardware of the interface and software during the automatic batch execution of the mapping process according to the method of the invention based on sequence data and parameters that are provided in advance, as described with reference to Embodiments 1 and 2. In the figure, the rectangles of thin lines indicate the processes of the interface, while the rectangle of bold lines indicates a process of software.

FIG. 16 shows a flowchart indicating the association between the processes performed by the hardware of the interface and software during the mapping process through the interactive interface described in Embodiment 3, in which the method of the invention is implemented. In the figure, the rectangles of thin lines indicate the processes of the interface, while the rectangle of bold lines indicates a process of software.

(Usefulness in Industry)

In order to take full advantage of the information contained in the cDNA sequences and genome sequences, a technology for mapping cDNA sequences to genome sequences is indispensable. As discussed in the related art section, mapping makes it possible to, for example, identify regions on genome sequences that correspond to genes, identify locations of particular genes on genome sequences, analyze promoter sequences, and identify the exon/intron structures of genes. When the genome sequence and the cDNA sequence are obtained from different individuals, SNPs can also be detected. The resultant data is essential for various biotechnology purposes, such as for drug discovery. Namely, the mapping technology provides a basis for many other technologies that utilize cDNA sequences and genome sequences.

In the field of education related to life science, when a course in which mapping of a cDNA sequences of a certain gene of interest to a genome sequence is conducted, the computer may be excessively burdened if a large number of students perform the mapping process simultaneously. This problem can be addressed by the method of the invention, which enables a mapping process to be performed on a small-scale, inexpensive computer. Thus, the invention provides a low-cost environment for carrying out such a course.

1. A method that maps a cDNA sequence to a genome sequence, which comprises:
entering a cDNA sequence;
dividing said cDNA sequence into K-base-length partial sequences;
dividing a genome sequence that is to be compared with said cDNA sequence into K-base-length partial sequences;
associating the coordinates of a K-base-length partial sequence on said genome sequence and that of a partial sequence on said cDNA sequence if the K-base-length partial sequences match each other;
forming a pair (p, q) for any of said associated cordinates where p is the coordinate of a K-base-length partial sequence on said cDNA sequence, and q is the coordinate of the matching K-base-length partial sequence on said genome sequence;
constructing sequences of said pairs for each p where the values of q are in decreasing order in each of said sequences;
concatenating said sequences of pairs in the increasing order of said first element p;
extracting from said connected sequence a subsequence with the increasing order of said second element q;
associating K-base-length partial sequences on said cDNA sequence with the K-base-length partial sequences on said genome sequence with regard to the extracted subsequence;
extending the correspondences of said K-base-length partial sequences to the alignment of said cDNA sequence and said genome sequence; and
outputting the alignment of said cDNA sequence and said genome sequence.

2. The method that maps a cDNA sequence to a genome sequence according to claim 1, wherein said K-base length is not more than 30-base length.

3. The method that maps a cDNA sequence to a genome sequence according to claim 1, wherein the step of dividing the cDNA sequence into K-base-length partial sequences comprises taking said partial sequences at any positions in said cDNA sequences.

4. The method that maps a cDNA sequence to a genome sequence according to claim 1, wherein the step of dividing the genome sequence into K-base-length partial sequences is performed so that there is no overlap between the K-base-length partial sequences.

5. A method that maps a cDNA sequence to a genome sequence according to claim 1, comprising focusing only on a window on a genome sequence that has a width W, and further moving said window.

6. The method that maps a cDNA sequence to a genome sequence according to claim 1, wherein the step of outputting the associating information comprises outputting two-dimensional information including one axis on which the cDNA sequence is disposed and the other axis on which the genome sequence is disposed.

7. The method that maps a cDNA sequence to a genome sequence according to claim 1, wherein the step of associating the individual bases includes a process of correcting locations of splice sites so that intron sequences start with GT and end with AG.

8. A system that maps a cDNA sequence to a genome sequence, which comprises:
a genome sequence storing means in which a genome sequence is stored;
an input unit for entering a cDNA sequence;
dividing means for dividing said cDNA sequence that has been entered into K-base-length partial sequences;
a dividing means for dividing said genome sequence that is stored into K-base-length partial sequences with a length of K bases;
a comparison means for comparing the K-base-length partial sequences on said cDNA sequence with the K-base-length partial sequences on said genome sequence in order to identify the coordinates of one or more K-base-length partial sequence on said genome sequence that matches with the K-base-length partial sequences on said cDNA sequence;
a calculation means for forming a pair (p, q) for any of cordinates of said K-base-length partial sequnces on said cDNA sequence and said genome sequence that match each other, where p is the coordinate of the K-base-length partial sequence on said cDNA sequence and q is the coordinate of the K-base-length partial sequence on said genome sequence;
means for constructing for each p a sequence consisting of said pairs such that the values of q are in decreasing order;
means for concatenating the sequences of pairs in the increasing order of said first element p;
means for extracting from said concatenated sequence a subsequence with the increasing order of said second element q;
means for associating the K-base-length partial sequences of said cDNA sequence with the K-base-length partial sequences of said genome sequence with regard to the extracted subsequence;
means for extending correspondences of said K-base-length partial sequences to the alignment of said cDNA sequence and said genome sequence; and
output means for outputting the association between individual bases.

9. A display method for graphically displaying the result of mapping K-base-length partial sequences on a cDNA sequence to K-base-length partial sequences on a genome sequence, and for displaying a new result of the mapping process when one or more of parameters changes, using a method for mapping a cDNA sequence to a genome sequence, which comprises: entering a cDNA sequence; dividing said cDNA sequence into K-base-length partial sequences; dividing a genome sequence that is to be compared with said cDNA sequence into K-base-length partial sequences; associating the coordinates of a K-base-length partial sequence on said genome sequence and that of a partial sequence on said cDNA sequence if the K-base-length partial sequences match each other; forming a pair (p, q) for any of said associated coordinates where p is the coordinate of a K-base-length partial sequence on said cDNA sequence, and q is the coordinate of the matching K-base-length partial sequence on said genome sequence; constructing sequences of said pairs for each p where the values of q are in decreasing order in each of said sequences; concatenating said sequences of pairs in the increasing order of said first element p; extracting from said connected sequence a subsequence with the increasing order of said second element qg associating K-base-length partial sequences on said cDNA sequence with the K-base-length partial sequences on said genome sequence with regard to the extracted subsequence; extending the correspondences of said K-base-length partial sequences to the alignment of said cDNA sequence and said genome sequence; and outputting the aligmnment of said cDNA sequence and said genome sequence, wherein said K-base length is not more than 30-base length, or wherein the step of dividing the cDNA sequence into K-base-length partial sequences comprises taking said partial sequences at any positions in said cDNA sequences, or wherein the step of dividing the genome sequence into K-base-length partial sequences is performed so that there is no overlap between the K-base-length partial sequences, or comprising focusing only on a window on a genome sequence that has a width W, and further moving said window, or wherein the step of outputting the associating information comprises outputting two-dimensional information including one axis on which the cDNA sequence is disposed and the other axis on which the genome sequence is disposed, or wherein the step of associating the individual bases includes a process of correcting locations of splice sites so that intron sequences start with GT and end with AG.
